(19) United States
(12) Patent Application Publication
(10) Pub. No.: US 2011/0082410 A1
(43) Pub. Date: Apr. 7, 2011

(54) Method for Reducing Pain during Photodynamic Therapy

(75) Inventors: Albrecht Volker, Nuthetal (DE); Wolfgang Neuberger, Dubai (AE)

(73) Assignee: CeramOptec Industries Inc.

(21) Appl. No.: 12/896,551

(22) Filed: Oct. 1, 2010

Related U.S. Application Data
(60) Provisional application No. 61/247,817, filed on Oct. 1, 2009.

(51) Int. Cl. A61B 18/18

(52) U.S. Cl. 604/20

(57) ABSTRACT

The present invention provides a method for application of photodynamic therapy which results in a reduction of pain experienced by a patient during treatment. An irradiation pattern of short pulses is applied to a treatment site. The same total energy dosage and light intensity parameters for a specific application, as known in the state of the art is used, but the radiation is applied in a series of short pulses. In a preferred embodiment, radiation is applied with about 350 millisecond pulses, and each pulse is separated by about 100 to 500 millisecond intervals. Application of radiation in pulses rather than continuous wave application reduces pain during treatment without sacrificing the therapeutic effect.

### DOMESTIC PRIORITY UNDER 35 USC 119(E)

This application claims the benefit and priority of U.S. Provisional Application Ser. No. 61/247,817 filed Oct. 1, 2009, entitled “Method for Reducing Pain during Photodynamic Therapy” by Volker Albrecht and Wolfgang Neuberger, which is incorporated by reference herein.

### BACKGROUND OF THE INVENTION

1. Field of the Invention

The present invention relates to the field of photodynamic therapy (PDT) in general, while in particular, it relates to reduction of pain experienced by patients during photodynamic therapy procedure.

2. Information Disclosure Statement

Photodynamic therapy (PDT) is a well-accepted procedure for treating a variety of ailments. Of particular importance is PDT's success in fighting cancer, but it also has beneficial uses in many fields including cosmetic hair removal, ocular disease treatments and wound treatment. PDT's usefulness is evident in its ability to selectively target diseased cells while leaving healthy cells unaffected.

PDT treatments generally consist of three steps. The first step involves systemically or locally administering a photosensitizer or its precursor to a patient. Systemic administration involves injection of the photosensitizer into the bloodstream, whereas local administration may involve the use of a cream or lotion for dermatological application. Photosensitizers, such as porphyrins or chlorins, exhibit the characteristic that they are non-reactive unless exposed to light of certain wavelengths. The next step involves allowing the photosensitizer to preferentially accumulate in diseased tissue. Photosensitizers tend to be taken up by abnormally proliferative cells such as cancerous cells. After a sufficient period of time, the body flushes most of the photosensitizers, but proliferative cells retain them for a longer period. The result is that photosensitizers then substantially exist only in close proximity to abnormal cells. Photosensitizers applied locally, generally require less time to accumulate in diseased, hyperplasic tissue than those applied systemically. Finally, as a last step, the site is irradiated with light of a suitable wavelength. This serves to activate the photosensitizer and produce its cytotoxic effects. Because their destructive range is very small, the destructive effect is limited only to those cells that are close to the photosensitizers. Because only primarily cancerous or abnormally growing cells remain close to photosensitizers, these abnormal cells are destroyed and healthy tissue is spared.

Although PDT is an improvement over other treatments in many applications, and especially in cosmetic applications such as hair removal, it still suffers from some drawbacks. One such drawback is that some patients experience unpleasant and painful sensations during treatment. These sensations, which can arise from the destructive effect of the treatment or from thermal effects of the radiation, can range from mild discomfort to extreme pain in certain patients. Mainly two types of pain have been associated with PDT treatment-a stinging sensation during the treatment and a post treatment pain typical of inflammation. The stinging action is due to neurogenic mechanism. The severity of pain can depend on the light source; intensity used and lesion size and it can vary from patient to patient. The mechanism behind PDT induced pain is yet unknown. It could be due to local hyperthermia and cytotoxic effect.

The pain experienced by patient is also related to the size of the lesions and redness of the treated area (Sandberg Carin et al., Act Derm. Venereol, 86, 2006, pp 404-408). In the same study, Sandberg et al., tested pre-treatment with caspsaicin cream to reduce PDT induced pain in patient, but no pain relief was obtained.

During light exposure treatments, especially in dermatological treatments, patients can experience some dysesthesia, or abnormal unpleasant sensation on the treatment area. This sensation is often characterized as a pricking, stinging or itching that can exacerbate into an extremely painful sensation. Thermal sensations are also characteristic of PDT, which can range from slight warmth to a burning sensation. Pressure is yet another sensation that has been known to occur during the course of PDT. Generally, dysesthesia is difficult to predict and varies from patient to patient, though it generally is more intense early in the course of treatment and declines during the course of the procedure.

It has been shown that pain and other unpleasant sensations felt during PDT are not only due to thermal effects of radiation. For example, a burning sensation felt by the patient during PDT has been documented in treatments involving tumors pre-treated with Aminolevulinic Acid (ALA), a precursor to Haematoporphyrin, but not experienced in normal tissue exposed to the same radiation. As a result, these burning sensations, at least in some cases, are not solely due to tissue heating, but rather due to other processes at work during the treatment. (Wang, Ingrid, “Photodynamic Therapy and Laser-Based Diagnostic Studies of Malignant Tumors”, Doctoral Thesis, The Jubileum Institute, Department of Oncology, Lund University Hospital, 1999, pp 53-55).

In another study ALA and methyl-ester-ALA was compared, it was found that during illumination topical methyl-ester-ALA gave significantly less pain compared to ALA. The reason for this difference has yet to be confirmed by further studies, it could be due to the chemical itself or its method of formulation. It has also been suggested that ALA is actively transported into the peripheral nerve endings, triggering nerve stimulation, when illuminated. This can be one other reason for experiencing pain during PDT procedure (Sandberg et al.).

A variety of approaches for alleviating PDT-associated discomfort and pain have been attempted. One method consists of the application of local anesthesia, including infiltrating and spraying with lidocaine, or applying a topical solution for transdermal delivery of lidocaine and prilocaln, though this has generally not produced any significant improvement. As a result, this approach has not been typically used. Another approach that has appeared to be effective has been to spray the area of treatment with isotonic saline or water. In order not to compromise the oxygen supply by causing vasoconstriction, a temperature of 15-20° C. has been used. In the Wang case, the water had an immediate effect, reducing the sensation of pain from moderate to slight pain. Of course, this approach adds additional steps to the procedure, causes additional inconvenience and clean-up, and the presence of water may influence the effectiveness or tissue depth of the treatment especially when using radiation with high water absorption properties. Another method under study involves use of nerve block 10-15 minutes before irradiation to provide pain relief during topical PDT for extensive facial actinic keratoses. 1.5-2 mL of local anesthetic mepivacaine-adrenaline is used as nerve block for peripheral nerve in facial regions (J. Paoli et al. Clinical and Experimental Dermatology, 33, 2008, pp 559-564).

Application of pulsed radiation in PDT treatments has been used for activating the photosensitizer, but has not been incorporated into PDT for the purpose of reducing or eliminating pain. U.S. Pat. No. 5,643,334 by Eckhouse et al. describes a method for diagnosing and treating cancer with a combination of PDT and pulse heating. The method utilizes pulsed radiation in photodynamic therapy to more effectively control temperature and to use thermal effects to enhance the destructive effects of PDT on cancerous cells. Accelerated heating also serves to coagulate the blood vessels in a tumor, thus limiting the blood supply to the tumor. The method provides pulses having a duration ranging from 0.1 to 100 ms and a frequency ranging from 0.1 to 1 pulse/s. This method purports to increase the effectiveness of PDT, but does not claim to reduce discomfort or pain.

U.S. Pat. No. 6,860,879 Irion et al., discloses a light application unit for PDT and Photodynamic diagnosis in non-malignant diseases related to dental problem. Wherein, in one of example, a tubular channel is used to supply cool compressed air to provide relief from pain induced by PDT. Use of cool compressed air is also noted to be not effective in controlling pain.

In a study conducted by William et al., using delta-ALA for superficial basal cell carcinomas, pain induced by PDT treatment was assessed. In this study no significant pain was reported in case of using low irradiance of range 10-50 mW/cm² whereas for higher irradiances the known method of anesthetic was used. (“Irradiance-Dependent Photo-bleaching and Pain in δ-Aminolevulinic Acid-Photodynamic Therapy of Superficial Basal Cell Carcinomas” William et al., Clinical Cancer Research, 14, 2008, pp 4475-4483).

The presence of pain or discomfort can be a significant deterrent among those who could benefit from PDT treatments, especially those who suffer from minor illnesses or cosmetic problems such as unwanted hair. A method that preserves the effectiveness of PDT while reducing pain would be extremely useful. The prior art discloses no effective methods for reducing pain during PDT, and particularly discloses no methods for reducing pain without additional steps or methods. It would be very useful to provide a method for administering photodynamic therapy characterized by reduced pain without the addition of anesthetic procedures or additional pain relief methods.

A method is needed for PDT treatment that prevents/minimizes pain associated with the activation of the photosensitizer by irradiation. The present invention addresses this need without addition requirement.

### OBJECTIVES AND BRIEF SUMMARY OF THE INVENTION

It is an objective of the present invention to reduce or eliminate discomfort/pain felt by patients during photodynamic therapy treatments.

It is another objective of the present invention to provide a method that reduces or eliminates pain or discomfort in patients due to PDT without reducing the therapeutic effectiveness of PDT.

It is further objective of the present invention to provide a suitable radiation pattern having short pulses without changing other treatment parameters to reduce pain.

Briefly stated, the present invention provides a method for application of photodynamic therapy which results in a reduction of pain experienced by a patient during treatment. An irradiation pattern of short pulses is applied to a treatment site. The same energy dosage and light intensity parameters for a specific application as is known in the state of the art is used, but radiation is applied in a series of short pulses. In a preferred embodiment, radiation is applied with about 350 millisecond pulses, and each pulse is separated by about 100 to 500 millisecond intervals. Application of radiation in pulses rather than continuous wave application reduces pain during treatment without sacrificing the therapeutic effect.

The above and other objects, features and advantages of the present invention will become apparent from the following description.

### DETAILED DESCRIPTION OF PREFERRED EMBODIMENTS

Certain types of PDT treatment can be fairly painful and this pain is one of the drawbacks especially for treating minor illnesses or cosmetic problems, such as removal of unwanted hair, skin rejuvenation or acne treatment. The present invention discloses a method of photodynamic therapy (PDT) comprising the application of radiation to a treatment site in a series of short pulses. It has been found that applying PDT treatment with known effective parameters in short pulses rather than continuous radiation decreases or eliminates pain and discomfort felt by patients during these treatments. By using a of short pulse irradiation pattern pain receptors in the regions are not stimulated thus no pain is experienced using present invention. Moreover, the use of pulsed irradiation also reduced the temperature in the tissue being treated compared to continuous form. In addition, the effectiveness of pulsed PDT relative to traditional PDT has been equivalent. Applying treatment radiation in pulses, thusly, does not appear to have a deleterious effect on targeted tumor or cell destruction, in particular.

The total energy delivered to the treatment site and the intensity or power density of the treatment irradiation is predetermined as it would be in state of the art PDT treatments. Instead of applying the radiation continuously, as in standard practice, an irradiation pattern of short pulses is chosen (e.g., 10-400 ms on, 20-500 ms off) to activate a PDT drug. In a preferred embodiment, a method to reduce pain during photodynamic therapy comprises the steps of a) administering a photosensitizer agent to a treatment site; b) allowing sufficient time for said photosensitive agent to accumulate in the targeted cells; and c) applying radiation to said treatment site in a series of pulses. Radiation is applied with pulses of equal intensity and sharp peak intensity. Additionally, these pulses can be constant duration pulses or variable duration pulses with constant or variable intensity. Irradiation time for a short pulse pattern is equal to quasi-continuous irradiation while the treatment time is extended due to the non-irradiation time intervals. Using the same dosage and same intensity as state of the art PDT treatment, a drastic reduction in the pain level is observed during the treatment as the pain receptors are not activated by short pulse irradiation.

Any known photosensitizers or photosensitizer precursors can be used with the present invention. Examples include porphyrins such as Haematoporphyrin, chlorins, and bacteriochlorins. Photosensitizer precursors such as alanine and 5-Aminolevulinic Acid (ALA) can also be used with the present method. In a preferred embodiment, the photosensitizer agent used for reducing pain during photodynamic therapy is methylene blue, for example for hair removal treatments. Known methods of applying radiation and light source may also be used, including coherent and non-coherent radiation emitting devices such as lasers, lamps, and similar. Coherent and non-coherent radiation emitting devices emit suitable wavelengths to activate by irradiation the administered photosensitizer agents. Wavelengths are selected based on the absorption characteristics of the photosensitizer. The present invention applies to all methods of PDT and improves known methods by reducing a patient's pain or discomfort during known PDT procedures.

The nature of the radiation pulsing is varied as is needed to maximize a patient's comfort. Because of individual sensitivities, this pattern of pulses may be varied to achieve the desired level of pain reduction or discomfort. In a preferred embodiment, a specific pulse pattern is selected and maintained during treatment by a suitable irradiation pattern generator. Examples of such a pattern generator include a pulser, a scanner, or other means known in art. The pattern of the pulses for applying radiation can be controlled by manual or automatic control means. A variety of patterns can be chosen and used as needed. One example would be to periodically block the laser, creating constant power pulses with equal durations and pulse frequency. As an alternative, pulses with high intensity peaks may be used to deliver increased energy densities. Whether constant intensity pulses or pulse peaks are used, the length of the pulses and the pulse frequency can be constant, or can be varied according to a preselected pattern, or can be varied by the user during treatment itself.

In a preferred embodiment, a pattern is chosen to address the common instance where the level of pain is most intense during the early part of treatment and declines thereafter. In this case, a pattern is chosen wherein the pulse length or frequency is relatively low during the early portion of the treatment, and is gradually increased during later treatments. In another preferred embodiment, the physician can manually control the pulse pattern. In this case, the physician responds to feedback from the patient to determine the patient's comfort, and can lower pulse duration or frequency as needed to reduce or eliminate that discomfort. In this same embodiment, the user can increase duration/frequency to maximize the number and length of pulses while maintaining patient comfort level.

The present invention is further illustrated by the following examples, but is not limited thereby.

### Example 1

Pulsed PDT was shown to be effective without pain in a treatment for removal of unwanted hair using the photosensitizer methylene blue in combination with pulsed radiation. In this procedure, methylene blue is applied so that a concentration of the photosensitizer is located in the hair follicles. Radiation initiates a cytotoxic effect that acts to destroy the hair follicle.

Based on the absorption characteristics of the Methylene Blue, radiation having a wavelength of 665 nm was selected to deliver an energy density of 50 J/cm² to the treatment site. In order to enable short treatment duration, power density was set as high as 10 W/cm². A 2 Watt PDT-laser was used. In order to maintain the proper power density, the light spot area was set to be as small as 0.2 cm², having a radius of 0.252 cm. A patient with a relatively normal response to stimuli was chosen.

The treatment started with 5 s CW irradiation which was very painful. In order to deter mine the proper pulse length/frequency, treatment was attempted under various pulse lengths and frequencies. The parameters were modified in 5 trial steps to yield a useful protocol of the present invention for most patients. The parameters are shown in table 1 below.

TABLE 1

| TRIAL | NUMBER OF | PULSE ON | PULSE OFF | FREQUENCY | |
|---|---|---|---|---|---|
| N^{o} | PULSES | (s) | (s) | (Hz) | PAIN |
| 1 | 2 | 2.5 | 0.5 | 0.33 | Very high to unbearable |
| 2 | 5 | 1 | 0.5 | 0.67 | High |
| 3 | 10 | 0.5 | 0.5 | 1.00 | Intermediate to small |
| 4 | 14 | 0.35 | 0.5 | 1.18 | None |
| 5 | 14 | 0.35 | 0.1 | 2.22 | None |

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method to reduce pain during photodynamic therapy (PDT), comprising the steps of:
a. administering a photosensitizer agent to a treatment site;
b. allowing sufficient time for said photosensitive agent to accumulate in the targeted cells;
c. applying radiation to said treatment site in a series of pulses.

2. The method to reduce pain during PDT according to claim 1, wherein said radiation is applied with pulses chosen from a group consisting of equal intensity and sharp peak intensity.

3. The method to reduce pain during PDT according to claim 2, wherein said radiation is applied with pulses chosen from a group consisting of constant duration pulses and variable duration pulses.

4. The method to reduce pain during PDT according to claim 2, wherein said radiation is applied with pulses chosen from a group consisting of constant intensity pulses and variable intensity pulses.

5. The method to reduce pain during PDT according to claim 1, further comprising the step of: controlling a pattern of said pulses by a control means.

6. The method to reduce pain during PDT according to claim 5, wherein said control means is chosen from a group consisting of manual control means and automatic control means.

7. The method to reduce pain during PDT according to claim 1, wherein said treatment site is skin for the purpose of removing hair.

8. The method to reduce pain during PDT according to claim 1, wherein said treatment site is skin for the purpose of skin rejuvenation and acne treatments

9. The method to reduce pain during PDT according to claim 1, wherein said photosensitizer agent is methylene blue.

10. The method to reduce pain during PDT according to claim 1, wherein said radiation is selected to have a wavelength suitable to activate said administered photosensitizer agent.

11. The method to reduce pain during PDT according to claim 9 wherein said radiation is selected to have a wavelength of about 665 nm.

12. The method to reduce pain during PDT according to claim 1 wherein, said series of pulses are selected to have a rate of about 1 to 3 Hertz with a pulse length of about 0.2 to 0.5 sec.

13. The method to reduce pain during PDT according to claim 1 wherein, said radiation is selected to have a wavelength of about 665 nm, said radiation is provided at an energy density of about 50 J/cm², and said pulse rate is selected to be 1 to 3 Hertz with a pulse length selected to be of about 0.2 to 0.5 sec.
